(19) United States
(12) Patent Application Publication
(10) Pub. No.: US 2019/0084905 A1
(43) Pub. Date: Mar. 21, 2019

(54) NOVEL PROCESS FOR MANUFACTURING 2-CHLORO-3,3,3-TRIFLUOROPROPENE FROM 1,2-DICHLORO-3,3,3-TRIFLUOROPROPENE

(71) Applicant: HONEYWELL INTERNATIONAL INC., Morristown, NJ (US)

(72) Inventors: Haiyou WANG, Amherst, NY (US); Hsueh Sung Tung, Getzville, NY (US)

(73) Assignee: HONEYWELL INTERNATIONAL INC., Morristown, NJ (US)

(21) Appl. No.: 15/760,848

(22) PCT Filed: Sep. 16, 2016
(86) PCT No.: PCT/US16/52042

§ 371 Date: Mar. 16, 2018

Related U.S. Application Data
(60) Provisional application No. 62/219,162, filed on Sep. 16, 2015.

(51) Int. Cl. C07C 17/087; C07C 17/354; C07C 19/10; C07C 21/18

(52) U.S. Cl. CPC C07C 17/087; C07C 17/354; B01J 23/76; C07C 21/18; C07C 19/10

(57) ABSTRACT

The present disclosure relates to a process for preparing 2-chloro-3,3,3-trifluorpropene comprising: (a) hydrogenating 1,2-dichloro-3,3,3-trifluoropropene in the presence of a hydrogenation catalyst to form 1,1,1-trifluoro-2,3-dichloropropane and (b) dehydrochlorinating 1,1,1-trifluoro-2,3-dichloropropane in the presence of a dehydrochlorination catalyst to form 2-chloro-3,3,3-trifluorpropene.

### FIELD OF THE DISCLOSURE

The present disclosure relates to a novel process for making 2-chloro-3,3,3-trifluoropropene.

### BACKGROUND OF THE DISCLOSURE

Hydrofluoroolefins (HFOs), such as tetrafluoropropenes (including 2,3,3,3-tetrafluoropropene (HFO-1234yf)), are now known to be effective refrigerants, fire extinguishants, heat transfer media, propellants, foaming agents, blowing agents, gaseous dielectrics, sterilant carriers, polymerization media, particulate removal fluids, carrier fluids, buffing abrasive agents, displacement drying agents and power cycle working fluids. Unlike chlorofluorocarbons (CFCs) and hydrochlorofluorocarbons (HCFCs), both of which potentially damage the Earth's ozone layer, HFOs do not contain chlorine and, thus, pose no threat to the ozone layer. HFO-1234yf has been shown to be a low global warming compound with low toxicity and, hence, can meet increasingly stringent requirements for refrigerants in mobile air conditioning. Accordingly, compositions containing HFO-1234yf are among the materials being developed for use in many of the aforementioned applications.

The preparation of HFO-1234yf generally includes at least three reaction steps, as follows:
(i) (CX₂═CCl—CH₂X or CX₃—CCl═CH₂ or CX₃—CHCl—CH₂X)+HF→2-chloro-3,3,3-trifluoropropene (HCFO-1233xf)+HCl in a vapor phase reactor charged with a solid catalyst;
(ii) 2-chloro-3,3,3-trifluoropropene (HCFO-1233xf)+HF→2-chloro-1,1,1,2-tetrafluoropropane (HCFC-244bb) in a liquid phase reactor charged with a liquid hydrofluorination catalyst; and
(iii) 2-chloro-1,1,1,2-tetrafluoropropane (HCFC-244bb)→2,3,3,3-tetrafluoropropene (HFO-1234yf) in a vapor phase reactor.
   wherein X is independently selected from F, Cl, Br, and I, provided that at least one X is not fluorine.

Thus, 2-chloro-3,3,3-trifluoropropene (HCFO-1233xf) is a useful intermediate for making 2,3,3,3-tetrafluoropropene. Thus, it is beneficial to find another method of making 2-chloro-3,3,3-trifluoropropene. The present specification describes a method of making 2-chloro-3,3,3-trifluoropropene from a readily available raw material.

### SUMMARY

The present specification discloses a process for preparing 2-chloro-3,3,3-trifluorpropene comprising:
(a) hydrogenating 1,2-dichloro-3,3,3-trifluoropropene (HCFO-1223xd) in the presence of a hydrogenation catalyst to form 1,1,1-trifluoro-2,3-dichloropropane (HCFC-243db) and
(b) dehydrochlorinating 1,1,1-trifluoro-2,3-dichloropropane in the presence of a dehydrochlorination catalyst to form 2-chloro-3,3,3-trifluorpropene.

### DETAILED DISCLOSURE

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

The present specification describes two reactions for preparing 2-chloro-3,3,3-trifluorpropene. The first reaction is a hydrogenation of 1,2-dichloro-3,3,3-trifluoropropene to form 1,2-dichloro-3,3,3-trifluoropropane, and the second reaction is a dehydrochlorination of 1,2-dichloro-3,3,3-trifluoropropane to form 2-chloro-3,3,3-trifluorpropene.

Hydrogenation is a chemical reaction between molecular hydrogen and a compound in the presence or absence of a catalyst. The reaction is one in which hydrogen adds to a double or triple bond connecting two carbon atoms in the structure of the molecule.

In an embodiment, the hydrogenation step may be performed in a batchwise operation and in another embodiment it may be a performed in continuous or semicontinuous operation. Furthermore, in an embodiment the hydrogenation reaction may be a liquid phase reaction, and in another embodiment, the hydrogenation reaction is conducted in the vapor phase. In an embodiment, the hydrogenation reaction in the vapor phase, may be performed in one step, while in another embodiment, it may be conducted in two or more steps. In another embodiment, it consists of, at least two vapor phase reaction steps. Suitable reactors include batch reactor vessels and tubular reactors.

More specifically, in an embodiment the hydrogenation of reaction can be achieved at relatively high levels by the use of at least two reaction steps wherein the first step of reaction is conducted under conditions effective to achieve a first, relatively low rate of conversion to produce a first step reaction effluent, and at least a second step of reaction which is fed by at least a portion of said first step effluent and which is conducted under conditions effective to achieve a second rate of conversion higher than said first rate. The reaction conditions are, in an embodiment, controlled in each of the first and second and various steps in order to achieve the desired conversion in accordance with the present invention. By way of example, but not by way of limitation, conversion of the feed material may be controlled or regulated by controlling or regulating any one or more of the following: the temperature of the reaction, the flow rate of the reactants, the presence of diluent, the amount of catalyst present in the reaction vessel, the shape and size of the reaction vessel, the pressure of the reaction, and any one combinations of these and other process parameters which will be available and known to those skilled in the art in view of the disclosure contained herein. For example, to control heat management, the hydrogenation reaction may be performed in more than one step. Hydrogenation is an exothermic reaction, and dependent on the reaction, it may produce sufficient heat to adversely effect the catalyst used, such as deterioration of the catalyst. By conducting the reaction in various steps such as, by adding a limited amount of hydrogen or by controlling the pressure, e.g., the amount of heat released is controlled. In an embodiment, the reaction may be stopped and when the reaction vessel has cooled, it may be restarted, and the process is repeated.

Although hydrogenation may be conducted without a catalyst, in an embodiment, a hydrogenation catalyst is utilized. Examples of catalysts for use in the present reaction include, but are not limited to, finely divided metals such as cobalt, iron, nickel, copper, platinum, palladium, rhodium, ruthenium, iridium, osmium, silver, gold, and the like. Each of these hydrogenation catalysts may be supported or unsupported. Suitable catalyst supports include carbon, e.g., activated carbon, which may be acid washed or have low ash content, or both, metal halides, e.g. metal fluorides, such as aluminum fluoride, chromium fluoride, yttrium fluoride, lanthanum fluoride, magnesium fluoride, titanium fluoride, zirconium fluoride, and the like, silica, metal oxides, such as aluminum oxide, magnesium oxide, zinc oxide, chromium oxide, yttrium oxide, lanthanum oxide, titanium oxide, zirconium oxide, and the like, and metal oxyhalides, e.g., metal oxyfluorides, such as oxyfluorides of aluminum, chromium, yttrium, lanthanum, titanium, zirconium, magnesium, and the like.

In an embodiment, the catalysts used are finely divided metals, such as Group VIII metals, e.g., platinum, palladium and nickel, which metals are unsupported or supported on aluminum fluoride or a mixture thereof. Mixture of metals, for example, mixture of Group VIII metals, may be used as the catalysts. The metal-containing precursor used to prepare the catalyst is preferably a metal salt (e.g., palladium chloride). Other metals, including other Group VIII metals, may be added to the support during the preparation of the catalyst.

The supported metal catalysts may be prepared by conventional methods known in the art such as by impregnation of the carrier with a soluble salt of the catalytic metal (e.g., palladium chloride or rhodium nitrate) as described by Satterfield on page 95 of Heterogenous Catalysis in Industrial Practice, 2nd edition (McGraw-Hill, New York, 1991). Palladium supported on alumina is available commercially. Another suitable procedure for preparing a catalyst containing palladium on fluorided alumina is described in U.S. Pat. No. 4,873,381, which is incorporated herein by reference.

In a supported metal catalyst, the concentration of metal, e.g., palladium, on the support, is typically in the range of from about 0.01% to about 10% by weight based on the total weight of the catalyst and support and in an embodiment is in the range of about 0.1% to about 5% by weight based on the total weight of the catalyst and support. In an embodiment, the catalyst for the first step of the process is palladium on carbon in the above range, or palladium on aluminum in the above range.

The relative amount of hydrogen fed during contact with 1,2-dichloro-3,3,3-trifluoropropene in a reaction zone containing the hydrogenation catalyst is from about 1 mole of H₂ per mole of 1,2-dichloro-3,3,3-trifluoropropene to about 5 moles of H₂ per mole of 1,2-dichloro-3,3,3-trifluoropropene and in another embodiment is from about 1 mole of H₂ per mole of 1,2-dichloro-3,3,3-trifluoropropene to about 4 moles of H₂ per mole of 1,2-dichloro-3,3,3-trifluoropropene and, in still another embodiment, is from about 1 mole of H₂ per mole of 1,2-dichloro-3,3,3-trifluoropropene to about 2 moles H₂ per mole of 1,2-dichloro-3,3,3-trifluoropropene.

The reaction zone temperature for the catalytic hydrogenation of 1,2-dichloro-3,3,3-trifluoropropene is typically in the range of from about 50° C. to about 350° C. C and in another embodiment, is in the range of from about 100° C. to about 250° C. The pressure is typically in the range of from about 1 to about 100 psig, and in another embodiment, is in the range of from about 5 to about 50 psig. The contact time is typically in the range of from about 1 to about 450 seconds, and in another embodiment is in the range of from about 10 to about 120 seconds.

The effluent from the reaction zone typically includes unreacted hydrogen, unconverted 1,2-dichloro-3,3,3-trifluoropropene, and 1,2-dichloro-3,3,3-trifluoropropane. The 1,2-dichloro-3,3,3-trifluoropropane is separated from the effluent by techniques known in the art, such as by distillation. In addition, in an embodiment, 1,2-dichloro-3,3,3-trifluoropropene is also separated from the effluent by techniques known in the art, such as by distillation, and is recycled back to undergo additional hydrogenation to form 1,2-dichloro-3,3,3-trifluoropropane. In some embodiments, side reactions such as hydrodehydrochlorination of 1,2-dichloro-3,3,3-trifluoropropane take place, generating small amounts of HCl and 2-chloro-1,1,1-trifluoropropane and/or 3-chloro-1,1,1-trifluoropropane. The generated HCl is removed from the effluent by using water or caustic scrubbers. When water extractor is used, HCl aqueous solutions of various concentrations are formed. When caustic scrubber is used, HCl is neutralized as a chloride salt in aqueous solution. The acid-free stream is then dried by techniques known in the art, such as by using sulfuric acid scrubber, molecular sieve adsorption column, phase separator, etc, prior to the isolation of 1,2-dichloro-3,3,3-trifluoropropane product for next step and 1,2-dichloro-3,3,3-trifluoropropene raw material for recycle. It may also be advantageous to periodically regenerate the hydrogenation catalyst after prolonged use while in place in the reactor. Regeneration of the catalyst may be accomplished by any means known in the art, for example, by passing air or air diluted with nitrogen over the catalyst at temperatures of from about 200° C. to about 500° C., but in another embodiment from about 300° C. to about 400° C., for about 0.5 hour to about 3 days. In this embodiment this is followed by H₂ treatment at temperatures ranging from about 100° C. to about 400° C., preferably from about 200° C. to about 300° C.

The separated 1,2-dichloro-3,3,3-trifluoropropane next undergoes dehydrohalogenation in the presence of dehydrochlorination catalyst. Four types of dehydrochlorination catalysts may be utilized.

The first class of catalysts for the dehydrohalogenation is carbon solids. Carbon used as a catalyst may come from any of the following sources: wood, peat, coal, coconut shells, bones, lignite, petroleum-based residues and sugar. Commercially available carbons which may be used include those sold under the following trademarks: Barneby & Sutcliffe™, Darco™, Nucharm, Columbia JXN™, Columbia LCK™ Calgon™ PCB, Calgon™ BPL, Westvaco™, Norit™, Takeda™ and Barnaby Cheny NB™. Examples are those described in U.S. Pat. No. 4,978,649.

In one embodiment of the invention, carbon includes three dimensional matrix carbonaceous materials which are obtained by introducing gaseous or vaporous carbon-containing compounds (e.g., hydrocarbons) into a mass of granules of a carbonaceous material (e.g., carbon black); decomposing the carbon-containing compounds to deposit carbon on the surface of the granules; and treating the resulting material with an activator gas comprising steam to provide a porous carbonaceous material. A carbon-carbon composite material is thus formed.

Embodiments of carbon as catalysts include both non-acid washed and acid-washed carbons. In some embodiments of this invention, suitable carbon catalysts may be prepared by treating the carbon with acids such as HNO₃, HCl, HF, H₂SO₄, HClO₄, CH₃COOH, and combinations thereof. Acid treatment is typically sufficient to provide carbon that contains less than 1000 ppm of ash. Some suitable acid treatments of carbon are described in U.S. Pat. No. 5,136,113. In some embodiments, an activated carbon is dried at an elevated temperature and then is soaked for 8 to 24 hours with occasional stirring in 1 to 12 weight percent of HNO₃. The soaking process can be conducted at temperatures ranging from about room temperature to about 80° C. The activated carbon is then filtered and washed with deionized water until the washings have a pH greater than 4.0 or until the pH of the washings does not change. Finally, the activated carbon is dried at an elevated temperature.

In some embodiments, carbon is an activated carbon. It may be in bulk or in some embodiments of this invention, carbon is a non-acid washed activated carbon. In other embodiments of this invention, carbon is an acid washed activated carbon. The carbon can be in the form of powder, granules, or pellets, and the like. The carbon solid also may be graphite.

Thus, examples of carbon solids include graphite, carbon blacks, activated carbons, three-dimensional matrix carbonaceous materials.

A second class of catalysts for the dehydrochlorination reaction includes metal halides and mixtures thereof, including mono-, bi, and tri-valent metal halides and their mixtures/combinations. Component metals include, but are not limited to alkali metals (Group 1 metals), alkaline earth metals (Group 2 metals), and Groups 3, 4, 6, 7, 8, 9, 10, 11, 12, 13, 14 metals and lanthanides. Examples include, but are not limited to, lithium, sodium, potassium, cesium, magnesium, calcium, strontium, barium, iron, cobalt, nickel, copper, zinc, aluminum, gallium, indium, scandium, yttrium, lanthanum, chromium, titanium, cerium, tin, manganese, and the like. For example the metal ions used in the metal halides include, Li⁺, Na⁺, K⁺, Cs⁺, Mg²⁺, Ca²⁺, Sr²⁺, Ba²⁺, Fe²⁺, Co²⁺, Ni²⁺, Cu²⁺, Zn²⁺, Al³⁺, Ga³⁺, In³⁺, Sc³⁺, Y³⁺, La³⁺, Cr³⁺, Fe³⁺, Co³⁺, Ti⁴⁺, Zr⁴⁺, Ce⁴⁺, Sn⁴⁺, Mn⁴⁺, and the like. Component halogens include, but are not limited to, F⁻, Cl⁻, Br⁻, and I⁻. Examples of useful mono- or bi-valent metal halides include, but are not limited to, LiF, NaF, KF, CsF, MgF₂, CaF₂, LiCl, NaCl, KCl, CsCl, and the like. These catalysts may be either unsupported or supported. Useful supports include, but are not limited to, activated carbon, graphite, fluorinated alumina, and fluorinated graphite. The concentration of metal halide on the support, is typically in the range of from about 1% to about 50% by weight based on the total weight of the catalyst and in an embodiment is in the range of about 5% to about 20% by weight based on the total weight of the catalyst.

The third class of catalysts for the dehydrochlorination reaction is halogenated metal oxides and their mixtures. These halogenated metal oxide catalysts may include, but are not limited to, mono-, bi-, and tri-valent metal oxides and their mixtures/combinations. Component metals include, but are not limited to alkali metals (Group 1 metals), alkaline earth metals (Group 2 metals), and Groups 3, 4, 6, 7, 8, 9, 10, 11, 12, 13, 14 metals and lanthanides. Examples include, but are not limited to, lithium, sodium, potassium, cesium, magnesium, calcium, strontium, barium, iron, cobalt, nickel, copper, zinc, aluminum, gallium, indium, scandium, yttrium, lanthanum, chromium, titanium, cerium, tin, manganese, and the like. For example the metal ions used in the metal oxides include, Li⁺, Na⁺, K⁺, Cs⁺, Mg²⁺, Ca²⁺, Sr²⁺, Ba²⁺, Fe²⁺, Co²⁺, Ni²⁺, Cu²⁺, Zn²⁺, Al³⁺, Ga³⁺, In³⁺, Sc³⁺, Y³⁺, La³⁺, Cr³⁺, Fe³⁺, Co³⁺, Ti⁴⁺, Zr⁴⁺, Ce⁴⁺, Sn⁴⁺, Mn⁴⁺, and the like. The catalyst is either unsupported or supported on a substrate. Useful supports include, but are not limited to, activated carbon, graphite, silica, alumina, fluorinated alumina, fluorinated graphite, and the like. The concentration of halogenated metal oxide on the support, is typically in the range of from about 1% to about 50% by weight based on the total weight of the catalyst and in an embodiment is in the range of about 5% to about 20% by weight based on the total weight of the catalyst.

The fourth class of catalysts for the dehydrochlorination reaction is neutral, i.e., zero valent, metals, metal alloys and their mixtures. Useful metals include, but are not limited to, Pd, Pt, Ru, Rh, Fe, Co, Ni, Cu, Mo, Cr, Mn, Ag, Pd, Os, Ir, Pt, and the like and combinations of the foregoing as alloys or mixtures. Useful examples of metal alloys include, but are not limited to, SS 316, Monel 400, Inconel 825, Inconel 600, and Inconel 625. The catalyst may be unsupported or supported on a substrate. Non-limiting examples of support include activated carbon, metal oxides (such as alumina), metal halides, e.g., metal fluorides, and metal oxyhalides, e.g., metal oxyfluorides. In another embodiment, the metal supports are metal halides, e.g., metal fluorides, and metal oxyhalides, e.g., metal oxyfluorides. Examples of the metals that may be included in the metal oxyfluorides are Al, Cr, Ti, Zr, Mg, and the like. Non-limiting examples of metal fluorides include, but are not limited to AlF₃, CrF₃, TiF₄, ZrF₄, MgF₂, and the like. The concentration of metal on the support, is typically in the range of from about 0.01% to about 10% by weight based on the total weight of the catalyst and in an embodiment is in the range of about 0.1% to about 1% by weight based on the total weight of the catalyst.

In an embodiment, the catalyst for the second step of the process is palladium on carbon or palladium on alumina. In an embodiment, the amount of palladium is in the range of from about 0.01% to about 10% by weight based on the total weight of the catalyst and support and in another embodiment is in the range of about 0.1% to about 5% by weight based on the total weight of the catalyst and support. In another embodiment, the catalyst for the second step is activated carbon, which may have a metal ion thereon, such as aluminum and/or iron and the like; AlF₃, MgF₂, and CsCl/MgF₂, e.g., 10 wt % CsCl/MgF₂; fluorinated Cr₂O₃, fluorinated Al₂O₃, fluorinated MgO and fluorinated Cs₂O/MgO, such as fluorinated 10 wt % Cs₂O/MgO; Ni, including nickel alloys, such as nickel-chromium alloy, nickel-chromium alloy, and the like; stainless steel, and the like.

The dehydrochlorination reaction according to the present process can be carried out in any reactor made of a material that is resistant to reactants employed, especially to hydrogen chloride. As used herein, reactor refers to any vessel in which the reaction may be performed in either a batchwise mode, or in a continuous mode. Suitable reactors include batch reactor vessels and tubular reactors.

In one embodiment, the reactor is comprised of materials which are resistant to corrosion including stainless steel, Hastelloy, Inconel, Monel, gold or gold-lined or quartz. In another embodiment, the reactor is TFE, or PFA-lined.

The dehydrochlorination reaction is conducted in the vapor phase. It is conducted under effective conditions to dehydrochlorinate 1,1,1-trifluoro-2,3-dichloropropane to 2-chloro-3,3,3-trifluoropropene. The reaction is conducted under conditions to effect dehydrochlorination. For example, in an embodiment the reaction is conducted at a temperature ranging from about 200° C. to about to 600° C. and in another embodiment, from about 250° C. to about to about 550° C. and in third embodiment from about 300° C. to about 500° C.

In another embodiment the reaction is conducted under a pressure ranging from about 0 to about 200 psig and in another embodiment, from about 10 to about 150 psig and in a third embodiment from about 20 to about 100 psig.

Finally, in an embodiment, the temperature of the reaction ranges from about 200° C. to about 600° C., and at any one of the pressures indicated hereinabove, and other embodiments from about 25° C. to about 550° C. at any one of the pressures indicated hereinabove and in other embodiments from about 300° C. to about 500° C. at any one of the pressures indicated hereinabove. The effluent from the reaction comprises HCl, 2-chloro-3,3,3-trifluoropropene, and unconverted 1,1,1-trifluoro-2,3-dichloropropane. The 2-chloro-3,3,3-trifluoropropene is separated therefrom by processes known in the art, such as distillation, and the like.

The 1,1,1-trifluoro-2,3-dichloropropane is separated from the effluent by processes known in the art, such as by distillation, and the like and may be recycled back to undergo additional dehydrochlorination.

The reactor effluent may be fed to a caustic scrubber or to a distillation column to remove the by-product of HCl to produce an acid-free organic product which, optionally, may undergo further purification using one or any combination of purification techniques that are known in the art.

It may also be advantageous to periodically regenerate the dehydrochlorination catalyst after prolonged use while in place in the reactor. Regeneration of the catalyst may be accomplished by any means known in the art. One method is by passing oxygen or oxygen diluted with nitrogen over the catalyst at temperatures of about 200° C. to about 600° C. in an embodiment from about 350° C. to about 450° C. for about 0.5 hour to about 3 days followed by halogenation treatment at temperatures of about 25° C. to about 600° C. and in another embodiment from about 200° C. to about 400° C. for metal halide catalysts and for halogenated metal oxide catalysts or by reduction treatment at temperatures from about 100° C. to about 400° C., and in another embodiment from about 200° C. to about 300° C. for metallic catalysts.

The inventors have found that such highly desirable levels of conversion and selectivity, and particularly from feed streams as described herein, can be achieved by the proper selection of operating parameters, including, but not necessarily limited to, catalyst type, reaction temperature, reaction pressure, and reaction residence time. Preferred aspects of each of these parameters are described below.

The particular form of the catalyst can also vary widely. For example, the dehydrochlorination catalysts may contain other components, some of which may be considered to improve the activity and/or longevity of the catalyst composition. The catalyst may contain other additives such as binders and lubricants to help insure the physical integrity of the catalyst during granulating or shaping the catalyst into the desired form. Suitable additives, may include, by way of example but not necessarily by way of limitation magnesium stearate, carbon and graphite. When binders and/or lubricants are added to the catalyst, they normally comprise about 0.1 to 5 weight percent of the weight of the catalyst.

It is also contemplated that a wide variety of contact times for the preferred reactions of the present invention may be used. Nevertheless, in certain preferred embodiments, the residence time is preferably from about 0.5 seconds to about 600 seconds.

The 2-chloro-3,3,3-trifluoropropene prepared in accordance with the present process can be used to make 2,3,3,3-trifluoropropene, in accordance with known reactions. For example, the 2-chloro-3,3,3-trifluoropropene is hydrofluorinated in the presence of a hydrofluorination catalyst to form 2-chloro-1,1,1,2-tetrafluoropropane (HCFC-244bb), which in turn is dehydrochlorinated to form 2,3,3,3-tetrafluoropropene.

More specifically, the HCFO-1233xf prepared as described herein is converted to HCFC-244bb. In one embodiment, this step can be performed in the liquid phase in a liquid phase reactor, which may be TFE or PFA-lined. Such a process can be performed in a temperature range of about 70° C. to about 120° C. and about 50 to about 120 psig.

Any liquid phase fluorination catalyst may be used. A non-exhaustive list includes Lewis acids, transition metal halides, transition metal oxides, Group IVb metal halides, a Group Vb metal halides, or combinations thereof. Non-exclusive examples of liquid phase fluorination catalysts are an antimony halide, a tin halide, a tantalum halide, a titanium halide, a niobium halide, and molybdenum halide, an iron halide, a fluorinated chrome halide, a fluorinated chrome oxide or combinations thereof. Specific non-exclusive examples of liquid phase fluorination catalysts are SbCl₅, SbCl₃, SbF₅, SnCl₄, TaCl₅, TiCl₄, NbCl₅, MoCl₆, FeCl₃, a fluorinated species of SbCl₅, a fluorinated species of SbCl₃, a fluorinated species of SnCl₄, a fluorinated species of TaCl₅, a fluorinated species of TiCl₄, a fluorinated species of NbCl₅, a fluorinated species of MoCl₆, a fluorinated species of FeCl₃, or combinations thereof.

These catalysts can be readily regenerated by any means known in the art if they become deactivated. One suitable method of regenerating the catalyst involves flowing a stream of chlorine through the catalyst. For example, from about 0.002 to about 0.2 lb per hour of chlorine can be added to the liquid phase reaction for every pound of liquid phase fluorination catalyst. This may be done, for example, for from about 1 to about 2 hours or continuously at a temperature of from about 65° C. to about 100° C.

This hydrofluorination step of the reaction is not necessarily limited to a liquid phase reaction and may also be performed using a vapor phase reaction or a combination of liquid and vapor phases, such as that disclosed in U.S. Published Patent Application No. 20070197842, the contents of which are incorporated herein by reference. To this end, the HCFO-1233xf containing feed stream is preheated to a temperature of from about 50° C. to about 400° C., and is contacted with a catalyst and fluorinating agent. Catalysts may include standard vapor phase agents used for such a reaction and fluorinating agents may include those generally known in the art, such as, but not limited to, hydrogen fluoride.

2-Chloro-1,1,1,2-tetrafluoropropane (HCFC-244bb), is then transferred to another reactor wherein the 244bb is dehydrohalogenated. The catalysts in the dehydrochlorination reaction may be metal halides, halogenated metal oxides, neutral (or zero oxidation state) metal or metal alloy, or activated carbon in bulk or supported form. Metal halide or metal oxide catalysts may include, but are not limited to, mono-, bi-, and tri-valent metal halides, oxides and their mixtures/combinations, and more preferably mono-, and bi-valent metal halides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Component halogens include, but are not limited to, F⁻, Cl⁻, Br⁻, and I⁻. Examples of useful mono- or bi-valent metal halide include, but are not limited to, LiF, NaF, KF, CsF, MgF₂, CaF₂, LiCl, NaCl, KCl, and CsCl. Halogenation treatments can include any of those known in the prior art, particularly those that employ HF, F₂, HCl, Cl₂, HBr, Br₂, HI, and I₂ as the halogenation source.

When neutral, i.e., zero valent, metals, metal alloys and their mixtures are used. Useful metals include, but are not limited to, Pd, Pt, Rh, Fe, Co, Ni, Cu, Mo, Cr, Mn, and combinations of the foregoing as alloys or mixture. The catalyst may be supported or unsupported. Useful examples of metal alloys include, but are not limited to, SS 316, Monel 400, Inconel 825, Inconel 600, and Inconel 625. Such catalysts may be provided as discrete supported or unsupported elements and/or as part of the reactor and/or the reactor walls.

Preferred, but non-limiting, catalysts include activated carbon, stainless steel (e.g., SS 316), austenitic nickel-based alloys (e.g., Inconel 625), nickel, fluorinated 10% CsCl/MgO, and 10% CsCl/MgF₂. A suitable reaction temperature is about 300° C. to about 600° C. and a suitable reaction pressure may be between about 0 psig to about 200 psig. The reactor effluent may be fed to a caustic scrubber or to a distillation column to remove the byproduct of HCl to produce an acid-free organic product which, optionally, may undergo further purification using one or any combination of purification techniques that are known in the art.

Many aspects and embodiments have been described and are merely exemplary and not limiting. After reading the specification, skilled artisans appreciate that other aspects and embodiments are possible without departing from the scope of the invention.

Other features and benefits of any one or more of the embodiments will be apparent from the hereinabove detailed description and the claims.

The following examples are given as specific illustrations of the teachings of the present disclosure. It should be noted, however, that the present disclosure is not limited to the specific details set forth in the examples. In addition, it is to be noted that all of the following examples are prophetic.

### Examples 1 and 2: Two-Stage Hydrogenation of 1,2-dichloro-3,3,3-trifluoropropene

The reactors used in the following examples are two-stage reactors constructed from two sections of ¾″×32″ 316 SS tube, which can be separately heated to different temperatures.

Catalyst loading in the following examples is as follows:

Reactor 1: 0.5 g of catalyst (1 wt % Pd on 4-8 mesh carbon) diluted with 10 cc ⅛″ SS protruded packing, catalyst equally distributed throughout.

Reactor 2: 4.0 g of catalyst (1 wt % Pd on 4-8 mesh carbon) diluted with 20 cc ⅛″ SS protruded packing, catalyst equally distributed throughout.

### Example 1: Two-Stage Hydrogenation of 1,2-dichloro-3,3,3-trifluoropropene

Reactors 1 and 2 are heated to 120, and 200° C., respectively in hydrogen flow. 99% pure 1,2-dichloro-3,3,3-trifluoropropene (HCFO-1223xd) feed is then introduced to the Reactor 1 and then Reactor 2 at a flow rate of 30 g/h. The hydrogen flow is adjusted to achieve a H₂/1223xd mole ratio of 1.5. The hydrogenation reaction is continuously performed over a period of 200 hours. Samples are taken at various points along the series of reactors to follow the percent conversion and selectivity. After the first reaction stage, it is expected that the conversion is about 50% or higher; after the second reaction stage, it is expected that the conversion is about 95% or higher with selectivity to 1,1,1-trifluoro-2,3-dichloropropane (HCFC-243db) expected to be about 97% or higher.

### Example 2: Two-Stage Hydrogenation of 1,2-dichloro-3,3,3-trifluoropropene

1,2-dichloro-3,3,3-trifluoropropene is hydrogenated using the same reactor system as in Example 1 under the same conditions except that 0.5% Pd/alumina catalyst is loaded in both reactors. The hydrogenation reaction is continuously performed over a period of 200 hours. Samples are taken at various points along the series of reactors to follow the percent conversion and selectivity. After the first reaction stage, the conversion is expected to be about 40% or higher; after the second reaction stage, the conversion is expected to be about 95% or higher with selectivity to 1,1,1-trifluoro-2,3-dichloropropane (HCFC-243db) expected to be about 97% or higher.

### Examples 3-6 Dehydrochlorination of 1,1,1-trifluoro-2,3-dichloropropane (HCFC-243db)

The following dehydrohalogenation reactions are performed in a cylindrical ¾″×32″ 316 SS tube reactor. Heating is provided by inserting the reactor into an electric furnace. Process temperatures are recorded using a multi-point thermocouple placed inside the reactor and within the catalyst bed. The 243db feed is fed into the bottom of the vertically mounted reactor and vaporized before reaching the catalyst bed. Effluent gases are passed through a gas sampling valve to monitor the progress of the reaction using GC analysis.

### Example 3: Dehydrohalogenation of HCFC-243db Over Carbon-Based Catalysts

In Example 3, two kinds of activated carbons are used as dehydrochlorination catalysts. 20 cc of catalyst is used. 99% pure 243db (CF₃CHClCH₂Cl) feed is passed over each catalyst at a rate of 12 g/h at a temperature of 350° C. The reaction is run for 50 hours. The table hereinbelow lists the ion concentration on the activated carbon. It is expected that both activated carbons provide 1233xf selectivity higher than 70%. It is also expected that the activated carbon with lower concentration of Al³⁺ and Fe³⁺ would exhibit a much higher selectivity to 1233xf.

TABLE 1
243db dehydrochlorination over various activated carbons at 350° C.

| | Ion concentration, ppm |
|---|---|
| Catalyst sample no. | Al³⁺ + Fe³⁺ |
| 1 | <50 |
| 2 | 9550 |

### Example 4: Dehydrohalogenation of HCFC-243db Over Metal Halide Catalysts

In Example 4, three different metal halides, AlF₃, MgF₂, and 10 wt % CsCl/MgF₂ are used as dehydrochlorination catalysts. 20 cc of catalyst is used. 99% pure 243db (CF₃CHClCH₂Cl) feed is passed over each catalyst at a rate of 12 g/h at a temperature of 350° C. and at atmospheric pressure. The reaction is run for 50 hours. It is expected that all three metal halide catalysts provide 1233xf selectivity higher than 70%. It is also expected that the 10 wt % CsCl/MgF₂ catalyst would exhibit the highest selectivity to 1233xf.

### Example 5: Dehydrohalogenation of HCFC-243db Over Halogenated Metal Oxide Catalysts

In Example 5, four different fluorinated metal oxides are used as dehydrochlorination catalysts: fluorinated Cr₂O₃, fluorinated Al₂O₃, fluorinated MgO and fluorinated 10 wt % Cs₂O/MgO 20 cc of catalyst is used. 99% pure 243db (CF₃CHClCH₂Cl) feed is passed over each catalyst at a rate of 12 g/h at a temperature of 350° C. and at atmospheric pressure. The reaction is run for 50 hours. It is expected that all four fluorinated metal oxide catalysts provide a 1233xf selectivity higher than 70%. It is also expected that the fluorinated 10 wt % Cs₂O/MgO catalyst would exhibit the highest selectivity to 1233xf.

### Example 6: Dehydrohalogenation of HCFC-243db Over Metallic Catalysts

In Example 6, four different metal/metal alloys are used as dehydrochlorination catalysts: Ni mesh, SS 316 chips (stainless steel), Monel 400 chips (nickel-copper alloy), and Inconel 625 chips (nickel-chromium alloy). 20 cc of catalyst is used. 99% pure 243db (CF₃CHClCH₂Cl) feed is passed over each catalyst at a rate of 12 g/h at a temperature of 350° C. and at atmospheric pressure. The reaction is run for 50 hours. It is expected that all four metal/metal alloy catalysts provide a 1233xf selectivity higher than 90%.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

1. A process for preparing 2-chloro-3,3,3-trifluorpropene comprising:
(a) hydrogenating 1,2-dichloro-3,3,3-trifluoropropene in the presence of a hydrogenation catalyst to form 1,1,1-trifluoro-2,3-dichloropropane and
(b) dehydrochlorinating 1,1,1-trifluoro-2,3-dichloropropane in the presence of a dehydrochlorination catalyst to form 2-chloro-3,3,3-trifluorpropene.

2. The process according to claim 1 wherein the hydrogenation catalyst is a metal selected from palladium, platinum, rhodium, iron, cobalt, nickel, and copper, which metal is unsupported or supported

3. The process according to claim 1 wherein the hydrogenation catalyst is supported on oxyfluoride of Al, Cr, Ti, Zr, or Mg or on fluorides of Al, Cr, Ti, Zr, or Mg.

4. The process according to claim 2 wherein the hydrogenation was conducted at a temperature ranging from about 50° C. to about 350° C.

5. The process according to claim 2 where conducted at a temperature ranging from about 100° C. to about 250° C.

6. The process according to claim 1 wherein the dehydrochlorination catalyst is a carbon solid selected from graphite, carbon black, activated carbon or three-dimensional matrix of carbonaceous material.

7. The method according to claim 1 wherein the dehydrochorination catalyst is a metal halide wherein the metal is lithium, sodium, potassium, cesium, magnesium, calcium, strontium, barium, iron, cobalt, nickel, copper, zinc, aluminum, gallium, indium, scandium, yttrium, lanthanum, chromium, titanium, cerium, tin, or manganese or mixture thereof wherein said metal halide is unsupported or supported.

8. The method according to claim 1 wherein the dehydrochlorination catalyst is a halogenated metal oxide which is unsupported or supported, wherein the metal is lithium, sodium, potassium, cesium, magnesium, calcium, strontium, barium, iron, cobalt, nickel, copper, zinc, aluminum, gallium, indium, scandium, yttrium, lanthanum, chromium, titanium, cerium, tin, manganese or mixture thereof.

9. The method according to claim 6 wherein the catalyst is supported on activated carbon, graphite, silica, aluminum, fluorinated alumina or fluorinated graphite.

10. The method according to claim 1 wherein the dehydrochlorination catalyst is a metal of zero oxidation state or a metal alloy, wherein the metal is Fe, Co, Ni, Cu, Mo, Mn, Ag, Ru, Rh, Pd, Os, Ir or Pt and the metal alloy is an alloy of nickel or steel.

11. The process according to claim 1 wherein the dehydrochlorination reaction is conducted at a temperature ranging from about 200° C. to about 600° C.

12. The process according to claim 1 wherein the dehydrochlorination reaction is conducted at a pressure ranging from about 0 to about 200 psig.

13. The process according to claim 1 wherein the 2-chloro-3,3,3-trifluorpropene is further hydrofluorinated in the presence of a hydrofluorination catalyst to form 2-chloro-1,1,1,2-tetrafluoropropane which in turn is dehydrochlorinated in the presence of a dehydrochlorination catalyst to form 2,3,3,3-tetrafluoropropene.

14. The method according to claim 7 wherein the catalyst is supported on activated carbon, graphite, silica, aluminum, fluorinated alumina or fluorinated graphite.
